(19) United States
(12) Patent Application Publication
(10) Pub. No.: US 2021/0163993 A1
(43) Pub. Date: Jun. 3, 2021

(54) COMPOSITIONS AND METHODS FOR PRODUCTION OF PROTEINS

(71) Applicant: The Regents of the University of Michigan, Ann Arbor, MI (US)

(72) Inventors: Peter Louis Freddolino, Ann Arbor, MI (US); Scott Allen Scholz, Ann Arbor, MI (US); Xiaoxia Nina Lin, Ann Arbor, MI (US)

(21) Appl. No.: 17/052,308

(22) PCT Filed: May 2, 2019
(86) PCT No.: PCT/US2019/030367

§ 371 Date: Nov. 2, 2020

Related U.S. Application Data
(60) Provisional application No. 62/666,198, filed on May 3, 2018.

(51) Int. Cl. C12N 15/90; C12P 19/30; C12N 15/74

(52) U.S. Cl. CPC C12N 15/90; C12N 15/74; C12P 19/30

(57) ABSTRACT

Provided herein are compositions and methods for production of proteins in microbial systems. In particular, provided herein are compositions and methods for expressing exogenous genes from specific chromosomal locations of E. coli. Specifically, the disclosure provides an E. coli bacterium comprising a recombination target site and/or heterologous gene at one or more genomic positions comprising at least one expression enhancement sequence. Further disclosed is a method for expressing a gene of interest, comprising contacting a nucleic acid encoding said gene of interest with the bacterium.

### CROSS-REFERENCE TO RELATED APPLICATIONS

This application claims priority to U.S. provisional patent application Ser. No. 62/666,198, filed May 3, 2018, which is incorporated herein by reference in its entirety.

### STATEMENT REGARDING FEDERALLY SPONSORED RESEARCH OR DEVELOPMENT

This invention was made with government support under Grant No. GM097033 awarded by National Institutes of Health. The government has certain rights in the invention.

### FIELD

Provided herein are compositions and methods for production of proteins in microbial systems. In particular, provided herein are compositions and methods for expressing exogenous genes from specific chromosomal locations of E. coli at exceptionally high efficiency.

### BACKGROUND

Escherichia coli has been the pioneering host for recombinant protein production, since the original recombinant DNA procedures were developed using its genetic material and infecting bacteriophages. As a consequence, and because of the accumulated know-how on E. coli genetics and physiology and the increasing number of tools for genetic engineering adapted to this bacterium, E. coli is the preferred host when attempting the production of a new protein. Also, it is still the first choice for protein production at laboratory and industrial scales for an important number of proteins, because of its fast growth and simple culture procedures.

However, when searching for an ideal system for protein production, this bacterial species is clearly far from offering, in generic terms, optimal conditions for protein production and downstream. Plasmid loss and antibiotic-based maintenance, undesired chemical inducers of gene expression, plasmid/protein-mediated metabolic burden and stress responses, lack of post-translational modifications (including the inability to form disulphide bonds), no or poor secretion, protein aggregation and proteolytic digestion, endotoxin contamination and complex downstream are among the main obstacles encountered during protein production in E. coli.

Improved systems and methods for expressing proteins in E. coli are needed.

### SUMMARY

Provided herein are compositions and methods for production of proteins in microbial systems. In particular, provided herein are compositions and methods for expressing exogenous genes from specific chromosomal locations of E. coli designed to yield very high expression levels by virtue of the chromosomal context of the integration.

The compositions and methods described herein overcome limitations of prior E. coli expression systems of low levels of expression. The systems and methods described herein find use in commercial and clinical applications for which a high level of expression of genes of interest is desired (e.g., production of industrial, research, and pharmaceutical proteins).

For example, in some embodiments, provided herein is a E. coli bacterium comprising a recombination target site and/or heterologous genes at one or more genomic positions selected from, for example, nucleotides 281404 to 288692, 4189573 to U.S. Pat. Nos. 4,204,803, 4,128,474 to U.S. Pat. Nos. 4,165,334, 4,003,592 to U.S. Pat. Nos. 4,060,559, 3,949,128 to U.S. Pat. Nos. 3,980,248, 4,313,204 to 4318232, or 3387507 to U.S. Pat. No. 3,439,571 (using numbering from GenBank sequence accession U00096.3) or homologous regions in other genomes and sequences at least 90% homologous (e.g., at least 90%, 91%, 92%, 93%, 94%, 95%, 96%, 97%, 98%, or 99% homologous).

Further embodiments provide an E. coli bacterium comprising a recombination target site and/or heterologous gene at one or more genomic positions comprising at least one expression enhancement sequence. In some embodiments, the expression enhancement sequence is at least 50% (e.g., at least 50%, 60%, 65%, 70%, or more) GC nucleotides. In some embodiments, the recombination target site and/or heterologous genes is flanked on both sides by said expression enhancement sequence. In some embodiments, the expression enhancement sequence is at least 500 bp (e.g., at least 500, 600, 750, 1000, 2000, or more bp) per side.

The present disclosure is not limited to particular recombination target sites. In some embodiments, the recombination target site is a site-specific recombination target site (e.g., including but not limited to, FLP recombination target (FRT), LOX, attP/B recognition sites, gamma-delta resolvase site, lambda red mediated recombination, Tn3 resolvase site, Clustered regularly interspaced short palindromic repeats (CRISPR), or φC31 integrase target site). In some embodiments, the target site further comprises one or more elements selected from, for example, a reporter construct, a promoter, a repressor, a selectable marker or a purification tag gene. The present disclosure is not limited to particular promoters. In some embodiments, the promoter is an inducible promoter. Examples include, but are not limited to, a lac promoter, a tac promoter, or a T7 promoter. In some embodiments, the bacterium expresses a gene of interest inserted into the chromosome at the genomic position at a higher level relative to the level of expression of the gene interested into a different location on the chromosome (e.g., at least 1.5, 2, 5, 10, 20, 30, 50, or 100-fold higher).

Additional embodiments provide a kit or system, comprising: a) a bacterium described herein; and b) a recombination enzyme specific for the recombination target. In some embodiments, the kit or system further comprises one or more components selected from, for example, growth medium, growth container, a plasmid or linear nucleic acid for inserting a gene of interest incorporated between a site-specific recombination target sequence, or components for purification of a protein expressed from said gene of interest.

Yet other embodiments provide a method of expressing a gene of interest, comprising: a) contacting a nucleic acid encoding said gene of interest with a bacterium described herein under conditions such that the nucleic acid integrates into the chromosome of the bacterium at the genomic positions; and b) expressing the gene of interest. In some embodiments, the nucleic acid is on a plasmid in between a site-specific recombination target sequence. In some embodiments, the method further comprises the step of purifying a protein expressed from the gene of interest (e.g., using one or more of affinity purification for the purification tag, ion exchange chromatography, or size exclusion chromatography). In some embodiments, the gene of interest encodes a protein selected from, for example, a research protein, a pharmaceutical protein, or an industrial protein.

Still other embodiments provide a bacterium described herein for use in expressing a gene of interest.

Additional embodiments are described herein.

### DESCRIPTION OF THE FIGURES

FIG. 1 shows library construction and data acquisition for position-dependent transcriptional propensity mapping. A) mNeonGreen reporter is controlled by the TetO1 promoter. B) To produce the reporter library, randomly barcoded reporter constructs in complex with Tn5 are electroporated into cells and randomly integrated into the E. coli genome in parallel. C) Transposon footprinting pairs barcode sequence (orange) with integration location on the genome. D) The reporter library is grown in M9 RDM to OD 0.2. Total RNA and DNA are extracted.

FIG. 2 shows that genome-position dependent transcriptional propensity from random integration of a barcoded reporter is non-random. A) Autocorrelation of raw RNA/DNA ratio values for replicate 1. B) Autocorrelation of raw RNA/DNA ratio values for replicate 2. C) Reporter integration number for 1 kb windows throughout the genome. D) Correlation between replicates for calculated transcriptional propensity from 500 bp rolling median windows (Spearman ρ=0.91). E) Transcriptional propensity (over 500 bp median rolling windows) mapped to specific integration locations on the E. coli genome.

FIG. 3 shows that transcriptional propensity peaks correspond to ribosomal RNA operon and macrodomain boundaries. A) Transcriptional propensity signal (as in FIG. 2E) superimposed on other genomic features of interest. B) Correlation of transcriptional propensity and distance from the nearest rrs operon (Spearman ρ=−0.56). C) Lowess fit of transcriptional propensity with rm distance (Lowess Fraction=0.33

FIG. 4 shows correlation of transcriptional propensity with binding of abundant NAPs and nucleotide content. A) Correlation of transcriptional propensity with enrichment by Fis binding (500 bp rolling median, Spearman ρ=0.5) B). Correlation of transcriptional propensity with enrichment by H-NS binding (500 bp rolling median, Spearman ρ=−0.58). C) Correlation of transcriptional propensity with AT content (500 bp rolling mean, Spearman ρ=−0.59). D) Genome view of an H-NS silenced region and surrounding genomic context.

FIG. 5 shows expression from plasmids that were identical with the exception of the GC content flanking the reporter construct.

### DEFINITIONS

To facilitate an understanding of the present invention, a number of terms and phrases are defined below:

As used herein, the “expression enhancement sequence” refers to a nucleic acid that enhances expression of a gene or other nucleic acid when located in the vicinity of the expression construct comprising the gene or other nucleic acid to be expressed. In some embodiments, the expression enhancement sequence flanks one or more sides (e.g., 3′ or 5′ side) of the expression construct. In some embodiments, the expression enhancement sequence is at least 50% (e.g., at least 50%, 60%, 65%, 70%, or more) GC nucleotides. In some embodiments, the expression enhancement sequence is at least a portion of nucleotides 281404 to 288692, 4189573 to U.S. Pat. Nos. 4,204,803, 4,128,474 to U.S. Pat. Nos. 4,165,334, 4,003,592 to U.S. Pat. Nos. 4,060,559, 3,949,128 to U.S. Pat. Nos. 3,980,248, 4,313,204 to 4318232, or 3387507 to U.S. Pat. No. 3,439,571 (using numbering from GenBank sequence accession U00096.3 or homologs thereof) of the E. coli genome and sequences at least 90% (e.g., at least 90%, 91%, 92%, 93%, 94%, 95%, 96%, 97%, 98%, or 99% homologous).

As used herein the term, “in vitro” refers to an artificial environment and to processes or reactions that occur within an artificial environment. In vitro environments include, but are not limited to, test tubes and cell cultures. The term “in vivo” refers to the natural environment (e.g., an animal or a cell) and to processes or reaction that occur within a natural environment.

As used herein, the term “microbe” refers to a microorganism and is intended to encompass both an individual organism, or a preparation comprising any number of the organisms.

As used herein, the term “microorganism” refers to any species or type of microorganism, including but not limited to, bacteria, archaea, fungi, protozoans, mycoplasma, and parasitic organisms.

As used herein, the term “prokaryotes” refers to a group of organisms that usually lack a cell nucleus or any other membrane-bound organelles. In some embodiments, prokaryotes are bacteria. The term “prokaryote” includes both archaea and eubacteria.

As used, the term “eukaryote” refers to organisms distinguishable from “prokaryotes.” It is intended that the term encompass all organisms with cells that exhibit the usual characteristics of eukaryotes, such as the presence of a true nucleus bounded by a nuclear membrane, within which lie the chromosomes, the presence of membrane-bound organelles, and other characteristics commonly observed in eukaryotic organisms. Thus, the term includes, but is not limited to such organisms as fungi, protozoa, and animals (e.g., humans).

As used herein, the term “cell culture” refers to any in vitro culture of cells, including, e.g., prokaryotic cells and eukaryotic cells. Included within this term are continuous cell lines (e.g., with an immortal phenotype), primary cell cultures, transformed cell lines, finite cell lines (e.g., non-transformed cells), bacterial cultures in or on solid or liquid media, and any other cell population maintained in vitro.

As used herein, the term “sample” is used in its broadest sense. In one sense, it is meant to include a specimen or culture obtained from any source, as well as biological and environmental samples. Such examples are not however to be construed as limiting the sample types applicable to the present invention.

### DETAILED DESCRIPTION OF THE DISCLOSURE

Provided herein are compositions and methods for production of proteins in microbial systems. In particular, provided herein are compositions and methods for expressing exogenous genes from specific chromosomal locations of E. coli.

The bacterial nucleoid is a dense structure composed of DNA, RNA and proteins that excludes other abundant cellular machinery, such as ribosomes and RNA polymerase (RNAP), from its interior (Chai et al. 2014; Jin and Cabrera 2006; Bakshi, Choi, and Weisshaar 2015). Several studies have demonstrated that packing of the nucleoid is non-random and condition dependent. For example, chromosome conformation capture studies in multiple bacterial species have revealed segments of DNA that preferentially self-interact, called chromosome interaction domains (Lioy et al. 2018a; Le et al. 2013a; Marbouty et al. 2015; Wang et al. 2015). During exponential growth, RNAPs are also organized into tight foci on the nucleoid surface, actively transcribing ribosomal RNA operons (rrn) (Cabrera and Jin 2006a). Despite the specific localization of DNA and RNAP, previous findings have shown that gene expression from different genomic loci is roughly equivalent, except for the effect of gene dosage, which decreases from the origin of replication to the terminus during exponential growth (Beckwith, Signer, and Epstein 1966; Sousa, de Lorenzo, and Cebolla 1997). Higher gene dosage near the origin is a result of multiple replication initiation events before terminus replication and cell division (Cooper and Helmstetter 1968).

Experiments conducted during the course of development of embodiments of the present disclosure identified a number of regions of the E. coli chromosome that resulted in increased levels of expression of genes of interest relative to other chromosomal locations.

Accordingly, provided herein are compositions and methods for expressing genes of interest in E. coli by insertion in the chromosome at the identified locations (e.g., the locations described in Table 1 or sequence at least 90% homologous (e.g., at least 90%, 91%, 92%, 93%, 94%, 95%, 96%, 97%, 98%, or 99% homologous) to the regions described in Table 1) or inserted within expression enhancement sequences.

In some embodiments, the present disclosure provides expression enhancement sequences for expression of genes or interest. As described in Example 2, experiments conducted during the course of development of embodiments of the present disclosure identified GC content of the flanking sequence as a factor in levels of expression of a reporter gene. In some embodiments, the expression enhancement sequence is at least 50% (e.g., at least 50%, 60%, 65%, 70%, or more) GC nucleotides. In some embodiments, the recombination target site and/or heterologous genes is flanked on both sides by said expression enhancement sequence. In some embodiments, the expression enhancement sequence is at least 500 bp (e.g., at least 500, 600, 750, 1000, 2000, or more bp) per side.

In some embodiments, the present disclosure provides an E. coli bacterium engineered for expression of genes of interest at one or more of the locations recited in Table 1 or inserted within expression enhancement sequences. The present disclosure is not limited to particular methods for use in incorporating a nucleic acid encoding a gene of interest into the chromosome of the bacterium. In some embodiments, site-specific recombination is utilized. Site-specific recombination, also known as conservative site-specific recombination, is a type of genetic recombination in which DNA strand exchange takes place between segments possessing at least a certain degree of sequence homology. Site-specific recombinases (SSRs) perform rearrangements of DNA segments by recognizing and binding to short DNA sequences (sites), at which they cleave the DNA backbone, exchange the two DNA helices involved and rejoin the DNA strands. While in some site-specific recombination systems just a recombinase enzyme and the recombination sites is enough to perform all these reactions, in other systems a number of accessory proteins and/or accessory sites are also utilized.

Recombination sites are typically between 30 and 200 nucleotides in length and consist of two motifs with a partial inverted-repeat symmetry, to which the recombinase binds, and which flank a central crossover sequence at which the recombination takes place. The pairs of sites between which the recombination occurs are usually identical, but there are exceptions (e.g. attP and attB of λ integrase).

The present disclosure is not limited to particular site specific recombination systems. Examples include, but are not limited to, FLP/FLP recombination target (FRT), CRE/LOX, lambda-integrase (using attP/B recognition sites), gamma-delta resolvase (from the Tn1000 transposon), Tn3 resolvase (from the Tn3 transposon), lambda red mediated recombination (from phage lambda), CRISPR/CAS9, transposases (e.g., Tn5) and φC31 integrase (from the φC31 phage).

Flp-FRT recombination involves the recombination of sequences between short flippase recognition target (FRT) sites by the recombinase flippase(Flp)derived from the 2 plasmid of baker's yeast Saccharomyces cerevisiae. The 34 bp minimal FRT site sequence has the sequence 5′GAAGTTCCTATTCtctagaaaGtATAGGAACTTC3′ (SEQ ID NO:1) for which flippase (Flp) binds to both 13-bp 5′-GAAGTTCCTATTC-3′ (SEQ ID NO:2) arms flanking the 8 bp spacer, e.g., the site-specific recombination (region of crossover) in reverse orientation. FRT-mediated cleavage occurs just ahead from the asymmetric 8 bp core region (5′tctagaaa3′) on the top strand and behind this sequence on the bottom strand (See e.g., Zhu X D, Sadowski P D (1995). “Cleavage-dependent Ligation by the FLP Recombinase”. Journal of Biological Chemistry. 270 (39): 23044-54; herein incorporated by reference in its entirety).

Cre-Lox recombination is similar to the FLP/FRT system. The system comprises a single enzyme, Cre recombinase, which recombines a pair of short target sequences called the Lox sequences (ATAACTTCGTATA-NNNTANNN-TATACGAAGTTAT) (SEQ ID NO:3). The Cre enzyme and the original Lox site called the LoxP sequence are derived from bacteriophage P1 (See e.g., Sauer, B. (1987). “Functional expression of the Cre-Lox site-specific recombination system in the yeast Saccharomyces cerevisiae”. Mol Cell Biol. 7 (6): 2087-2096; herein incorporated by reference in its entirety).

Lambda-integrase (using attP/B recognition sites) is described, for example, in Landy, A. (1989). “Dynamic, Structural, and Regulatory Aspects of lambda Site-Specific Recombination”. Annual Review of Biochemistry. 58 (1): 913-41; herein incorporated by reference in its entirety. Additional phage and transposon systems are described, for example, in Stark, W. M.; Boocock, M. R. (1995). “Topological selectivity in site-specific recombination”. Mobile Genetic Elements. Oxford University Press. pp. 101-29; herein incorporated by reference in its entirety.

Lambda-red mediated recombination is described in Datsenko and Wanner 2000. “One-step inactivation of chromosomal genes in Escherichia coli K-12 using PCR products”. Proc Natl Acad Sci USA. 2000 Jun. 6; 97(12):6640-5; herein incorporated by reference in its entirety). By introducing a PCR product or plasmid containing regions for recombination at a specific genomic location, a genetic element may be permanently introduced at a specific location in a host genome.

Clustered regularly interspaced short palindromic repeats (CRISPR) are segments of prokaryotic DNA containing short, repetitive base sequences. These play a key role in a bacterial defense system, and form the basis of a genome editing technology known as CRISPR/Cas9 that allows permanent modification of genes within organisms (See e.g., Zhang F, Wen Y, Guo X (2014). “CRISPR/Cas9 for genome editing: progress, implications and challenges”. Human Molecular Genetics. 23 (R1): R40-6; herein incorporated by reference in its entirety). By delivering the Cas9 nuclease complexed with a synthetic guide RNA (gRNA) into a cell, the cell's genome can be cut at a desired location, allowing existing genes to be removed and/or new ones added.

In some embodiments, transposases (e.g., Tn5) and transopons are utilized for incorporation of genes of interest (See e.g., Example 1 and Reznikoff W S (March 2003). “Tn5 as a model for understanding DNA transposition”. Molecular Microbiology. 47 (5): 1199-206; herein incorporated by reference in its entirety).

In some embodiments, the recombination enzyme specific for the chosen target site is provide on a plasmid or chromosomal location (e.g., under the control of an inducible promoter).

In some embodiments, target sites on the E. coli chromosome further comprise additional elements useful for expression of a gene of interest. Examples include, but are not limited to, promoters (e.g. inducible or constitutive promoters), repressors or operators for control of the promoter, reporter constructs, or a gene encoding a protein purification tag.

In some embodiments, reporter constructs are utilized to measure or identify expression or the gene or nucleic acid of interest. The present disclosure is not limited to particular reporter constructs. Examples include, but are not limited to, fluorescent reporters (e.g., green fluorescent protein or red fluorescent protein), beta-galactosidase, luciferase, chloramphenicol acetyltransferase etc. (See e.g., Ghim et al., BMB Rep. 2010 July;43(7):451-60; herein incorporated by reference in its entirety) for a review.

The present disclosure is not limited to particular promoters. In some embodiments, the promoter is an inducible promoter. Examples include, but are not limited to, a lac promoter, a tac promoter, or a T7 promoter (See e.g., Dubendorff J W, Studier F W (1991). “Controlling basal expression in an inducible T7 expression system by blocking the target T7 promoter with lac repressor”. Journal of Molecular Biology. 219 (1): 45-59; deBoer H. A., Comstock, L. J., Vasser, M. (1983). “The tac promoter: a functional hybrid derived from trp and lac promoters”. Proceedings of the National Academy of Sciences USA. 80 (1): 21-25; each of which is incorporated by reference in its entirety).

Examples of protein purification tags include, but are not limited to, histidine (His) tag, glutathione S-transferase or maltose-binding protein.

In some embodiments, the bacterium described herein is provided in the form of a kit or system, comprising, in one or more containers, one or more or all of a bacterium described herein, a recombination enzyme specific for the recombination target on the bacterial chromosome (e.g., provided as a plasmid or incorporated into the genome of the bacterium), growth medium, growth container, inducers or enhancers of an inducible promoter, a plasmid for inserting a gene of interest in between a site-specific recombination target sequence, or components for purification of a protein expressed from said gene of interest.

In operation, the present disclosure provides a method of expressing a gene of interest at a specific location (e.g., those described in Table 1 or sequences with a high GC content as described above) on an E. coli chromosome. In order to express a gene of interest, one introduces a nucleic acid encoding the gene of interest (e.g., on a plasmid or other vector) into a bacterium described herein. In some embodiments, the gene is inserted in between target recombination sites on the plasmid or vector. The vector comprising the gene of interest is transferred into the bacterium (e.g., using transfection or other technique). The recombination enzyme is provided (e.g., by inducing its expression) such that the gene of interest incorporates into the chromosome at the desired location using site-specific recombination.

In some embodiments, bacteria are cultured (e.g., using a liquid culture system or on a culture medium) in order to produce protein encoded by the gene of interest. Bacteria are grown for the desired length of time in order to produce adequate amounts of protein. In some embodiments, growth is monitored (e.g., via light microscopy or visible observation). In some embodiments, culture medium is replenished one or more times during culture.

In some embodiments, the bacterium expresses a gene of interest inserted into the chromosome at the genomic position at a higher level relative to the level of expression of the gene interested into a different location on the chromosome (e.g., at least 1.5, 2, 5, 10, 20, 30, 50, or 100-fold higher).

In some embodiments, following expression, a protein expressed from the gene of interest is purified. Following lysis of cells, proteins of interest are purified using any suitable method (e.g., one or more of affinity purification for a purification tag, ion exchange chromatography, or size exclusion chromatography).

The present disclosure is not limited to particular genes of interest. In some embodiments, the gene of interest encodes a protein for research or screening use, a pharmaceutical protein, or an industrial protein.

### Experimental

The following example is provided in order to demonstrate and further illustrate certain preferred embodiments and aspects of the present invention and are not to be construed as limiting the scope thereof.

### Example 1

### Materials and Methods

### Reporter Construct Design

The mNG coding sequence was obtained from Allele Biotech. mNG was under the TetR1 promoter and the B0030 ribosome binding site, which is predicted to have 30-fold lower translation initiation rate than the highest rate of a native gene in E. coli. (Kosuri et al. 2013b; Espah Borujeni, Channarasappa, and Salis 2014). Upstream of the mNG cassette, an FRT-flanked kanamycin resistance cassette amplified from the Keio collection was introduced in the divergent orientation relative to mNG (Baba et al. 2006). Directly downstream of the mNG coding sequence, an Illumina i5 adapter primer complement sequence and an AscI recognition site for later barcoding of the integration construct were inserted. The reporter and antibiotic cassettes are flanked by the strong bidirectional terminators L3S2P21 and ECK120026481 (Y.-J. Chen et al. 2013b). Finally the entire cassette is flanked by mosaic ends (MEs) to allow for binding to Tn5 transposase. The ME-flanked construct was modified to remove two PvuII restriction sites in order to allow for PvuII digestion of the plasmid pSAS31 and release the integration construct for Tn5 transposase binding in vitro.

### Strain Background Design

MG1655 (CSGC 7740) was obtained from the Coli Genetic Stock Center (CGSC, Yale) (Blattner 1997). P1 vir transduction was used to introduce the Z1 cassette from MG1655 Z1 malE (Addgene plasmid #65915) into MG1655. This MG1655 Z1 strain was then transformed with the lambda red plasmid pSIM5. The primers BT1promCh F and BT1promCh R were used to amplify the mCherry and ampicillin resistance cassette from pBT1-proD-mCherry (Addgene plasmid #65823). The mCherry cassette was then integrated into a site directly downstream from yihG using lambda red recombination to produce ecSAS17 (MG1655 malE::Z1 mCherry+AmpR). The mCherry integration was confirmed by genotyping and the transduction of the Z1 cassette by observing TetR-mediated repression of mNG compared to a blank MG1655 strain. ecSAS17 was then transformed with the pBAD-Flp plasmid (see below) to provide the starting strain for library generation.

### Large-Scale Plasmid Barcoding

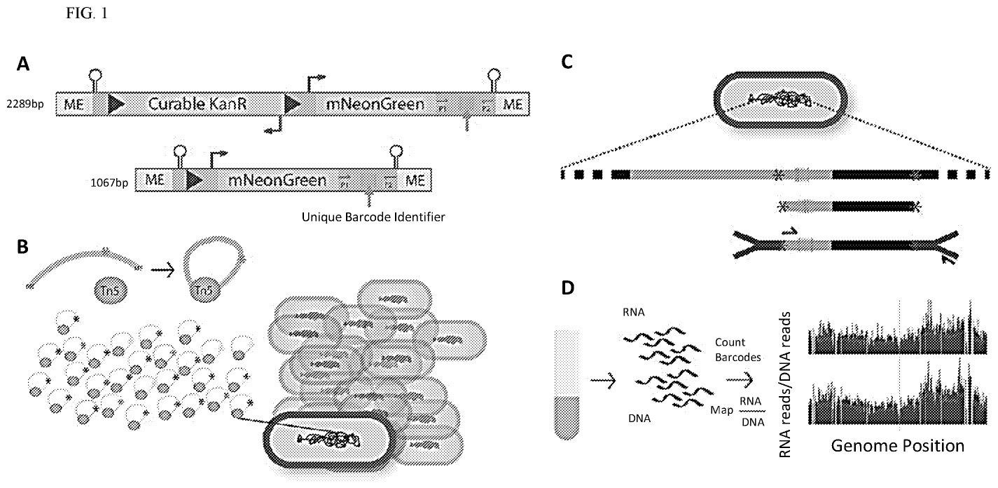

pSAS31 was digested with the restriction enzyme AscI. Primers were used to introduce the barcode and amplify the entire plasmid by PCR (FIG. 1). The resulting fragment was digested by DpnI and AscI, and then ligated with T4 ligase overnight at 14° C. The reaction was quenched with EDTA. The Hanahan procedure was scaled up to transform chemically competent cells with the ligated plasmid (Hanahan, Jessee, and Bloom 1991). Cells were recovered in SOC for one hour at 37° C. before removing an aliquot for transformation efficiency counts and adding kanamycin for 8 h liquid selection at 37° C. Cells were then pelleted for 7 minutes at 4600×g and snap frozen in liquid nitrogen. To obtain the plasmid, snap-frozen cells were resuspended in lysis buffer for plasmid miniprep. pBADpCP20 plasmid construction The pCP20 plasmid causes over 90% of cells with an FRT-flanked kanamycin resistance cassette to lose resistance even at the uninduced 28° C. temperature, presumably due to leaky expression of Flp recombinase. Since Flp recombinase leaking from the pCP20 plasmid reduced transposon integration efficiency, probably due the removal of the KanR cassette soon after integration and prior to liquid-phase selection, the PR temperature sensitive promoter on pCP20 was replaced with the arabinose-inducible promoter pBAD and repressor araC gene. The modified pBADpCP20 plasmid did not cause detectable loss of the KanR cassette under uninduced conditions.

### Tn5 Integration of Barcoded Reporter Constructs

To generate stable transposomes for electroporation into the target strain, barcoded pSAS31 plasmid was digested with PvuII for one hour at 37° C. and fragments were separated on a 0.8% agarose gel. The band corresponding to the integration fragment size was cut out of the gel and purified. 200 ng/μl fragment was then incubated with 2 μl Tn5 transposase and 1 μl glycerol according to the manufacturer's instructions. After 30 minutes incubation at room-temperature, the mixture was stored at −20° C. Electrocompetent cells were prepared using ecSAS17 with chloramphenicol included in the growth medium in order to maintain the pBADpCP20 Flp recombinase plasmid. 1 μl of the Tn5-DNA complex was mixed with 50 μl of fresh electrocompetent cells. Four separate electroporations were carried out at 1800 kV and immediately resuspended in 1 mL of 30° C. SOC medium. Each reaction was pooled into SOC medium including chloramphenicol and incubated at 30° C. for 1.5 hours. An aliquot for plating was removed from the recovery medium before adding Kanamycin. Liquid selection proceeded for 16 hrs at 30° C. After liquid selection, all cells were pelleted at 4600×g for 7 minutes. Cells were then resuspended in 30 mL 15% glycerol, pipetted into 30 1 mL aliquots and snap frozen in a dry-ice ethanol bath before storage of the transposon library at −80° C. (Girgis et al. 2007b). According to colony forming unit counts from plating after recovery, 609,000 cells were uniquely transformed and maintained pBAD-Flp, as indicated by resistance to kanamycin and chloramphenicol.

Pairing Integration Site with Barcode Via Transposon Footprinting

Cells from one aliquot of the transposon library were recovered in 5 mL SOC for 30 minutes at 30° C. with shaking. Genomic DNA was isolated from the library using the Qiagen Blood and Tissue kit for Gram negative bacteria. 1 μ g of the resulting DNA was digested with either CviAII or CviQI restriction enzymes (each has a different 4 bp cut site but leaves compatible overhangs). An annealed Y-linker that complements the overhangs was ligated to the digested DNA fragments with T4 DNA ligase for 10 minutes. The reaction was quenched with EDTA. The DNA from the ligation mix was purified with Axygen AxyPrep Mag PCR cleanup beads at a 0.9:1 bead to DNA ratio to remove unligated Y-linker. The resulting DNA was amplified by PCR using the primers that bind within the transposon and on the Y-linker to amplify transposon-genomic DNA specific fragments. Illumina adapters were added to the resulting fragment by PCR.

### Full-Scale Genome Profiling Procedure

The cryopreserved transposon library was scraped into 1 mL of M9-EZrich medium and diluted into 50 mL of M9-EZrich 1% Arabinose+0.4% glycerol+chloramphenicol in a baffled 125 mL flask to achieve OD of 0.0031 600 nm. The flask was incubated at 30° C. for 8 hours with shaking at 225 rpm to allow Flp recombinase to excise the kanamycin resistance cassette. Cells were then pelleted at 4600×g for 7 minutes and resuspended in 50 mL PBS. In parallel, an aliquot of the culture was diluted and plated on LB-kanamycin and LB plates to determine the fraction cell that permanently lost kanamycin resistance (<93%). Cells were pelleted again and resuspended in 10 mL M9RDM. Cells were then diluted into 100 mL of M9RDM+100 ng/mL Anhydrotetracycline (aTc) to a final 0.0031 OD600. The culture was incubated at 37° C. until an OD600 of 0.2 was reached (about 6 hours) to allow induction of the transposon-born reporter construct. The entire flask was then immediately transferred to an ice-slurry bath. Three aliquots of 5 mL were then pelleted at 6600×g for 3 minutes and snap-frozen in a dry-ice ethanol bath to allow harvest of genomic DNA. In parallel, three additional aliquots of 5 mL of the culture was rapidly mixed with 25 mL Bacteria RNA protect reagent (Qiagen) and frozen according to the manufacturer's instructions to allow harvest of RNA from matched samples of the growing library. All samples were then stored at −80° C.

### Nucleic Acid Processing and Sequencing

Genomic DNA (gDNA) from harvested samples was extracted following the Qiagen Blood and Tissue kit instructions. 1 μg of gDNA was then digested for 1 hour with CviQI. The resulting DNA was purified with PCR cleanup kit and eluted into 0.1×TE. The DNA was then lightly amplified with primers flanking the barcode for eight cycles using Q5 polymerase, resulting in a 186 bp fragment. The DNA from the PCR mix was purified with Axygen AxyPrep Mag PCR cleanup beads at a 0.9:1 bead to DNA ratio to remove unincorporated primers. RNA from the exponentially growing cells was extracted following the Qiagen RNeasy Bacterial RNA protect protocol including on-column DNaseI treatment. 1 g of the resulting RNA and a single reverse primer were used for first strand synthesis with the NEB ProtoscriptII First Strand cDNA kit using the manufacturer's instructions, and the resulting cDNA was stored at −20° C. No-polymerase controls (−RT) were included. 20 μl of the cDNA or 5 μl of cDNA reaction mixture was used for a 50 μl minimal-cycle PCR amplification using NEB Q5 hotstart polymerase, following the manufacturer's instructions with the following modifications: NEB i5xx or i7xx primers were used to add Illumina adapter sequences. EvaGreen dsDNA dye to a final 1× concentration was added to each reaction. 10 μl of each reaction (including −RT controls) were then monitored for qPCR fluorescence signal during PCR amplification. The remaining 40 μl of each reaction was then amplified with the number of PCR cycles corresponding to 25% of the maximum fluorescence observed in the 10 μl qPCR pilot reaction. The cycle threshold for the −RT cDNA controls were verified to be at least 7 cycles greater than the standard cDNA samples (indicating background from DNA contamination of less than 1%). Each 40 μl PCR reaction was then purified with 90 μl of Axygen MAG-S1 beads and eluted in 0.1×TE. The purified DNA was submitted to the University of Michigan sequencing core for sequencing on a NextSeq 550.

### Results

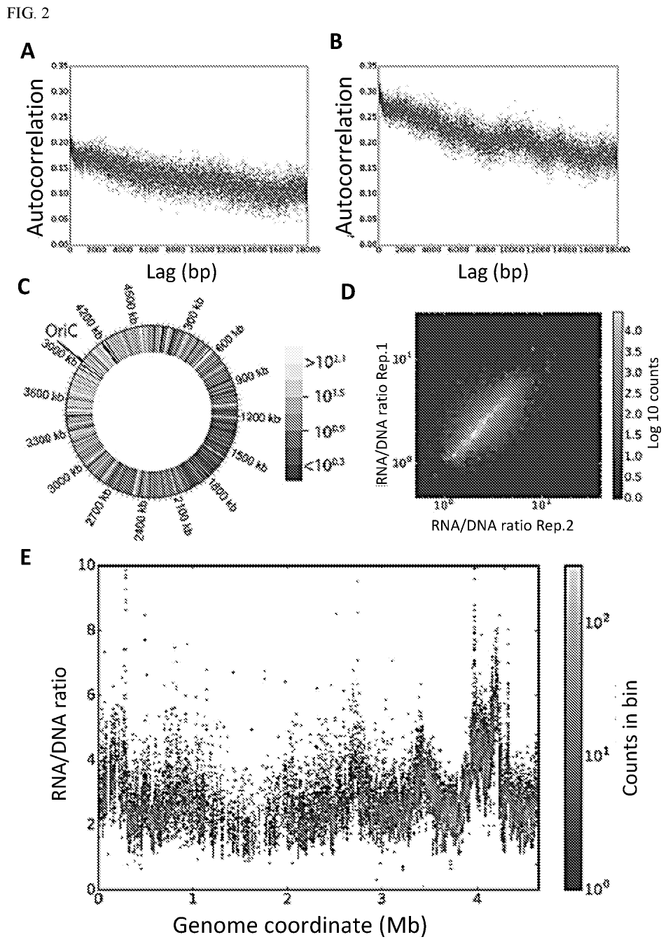

Using a modification of transposon footprinting (Girgis et al. 2007a), 144,000 random integration reporter barcodes were paired were to their genomic integration locations in E. coli. Higher densities of reporter integrations were present around the origin of replication (Ori), likely due to higher Ori-Terminus chromosome copy number during exponential phase growth at the time of transformation (FIG. 2C). On average, one unique barcoded integration is present every 33 bp, greatly exceeding the overall resolution of position-dependent expression variation achieved by any previous works. To obtain information of the amount of RNA transcript produced per unit DNA present in a growing library (henceforth referred to as the transcriptional propensity at that location), matched RNA and DNA samples were extracted from exponentially growing samples of the reporter library during induction with anhydrotetracycline (aTc). RNA and DNA reporter barcodes were sequenced and mapped to the corresponding genomic locations determined by transposon footprinting. Autocorrelation analysis was used as a quantitative measure of correlation between raw transcriptional propensities as the bp distance between insertion sites increases to determine whether reporters integrated into similar locations also exhibit similar expression. It was demonstrated that neighboring integrations have a high similarity in raw transcriptional propensity, which generally decreases as bp distance increases. Therefore, reporter transcription is non-random and dependent on integration location (FIG. 2A, B). Autocorrelation of transcriptional propensity between neighboring integrations was far higher here as compared to a previous massively-parallel reporter integration study, likely due to the higher integration density (Akhtar et al. 2013). The RNA per DNA barcode values were smoothed by assigning the median from a 500 bp window around each integration site with a minimum of three reporter integrations within the window. Data from two replicates from the transposon library (FIG. 2D, Spearman ρ=0.915) were quantile normalized and averaged. The resulting transcriptional propensities were mapped onto the E. coli genome. Transcriptional propensity variation appears roughly waveform at the whole-genome scale (FIG. 2E). Several sharp troughs are also apparent, independent of the overall waveform. Transcriptional propensities are not a result of gene dosage resulting from high Ori-Ter ratios during exponential phase growth or from differing representation of a library member because all transcriptional propensities are reported as RNA per DNA ratios. Consistent with previous works that found little to no impact of barcode sequences on expression, there is very low correlation of transcriptional propensity with barcode GC content (Spearman ρ=0.13 and 0.15 for reach replicate, respectively), which was eliminated by the median windowing described above (Spearman ρ=0.013). Two sites where transcriptional propensity from reporters within a gene differed from the surrounding neighborhood that could be attributed to gene knockout effects were identified (rep and bioH), but these were the exception, rather than the rule. It is contemplated that integrations in genes that would globally affect transcriptional propensity also result in a competitive disadvantage during growth and were therefore lost from the library.

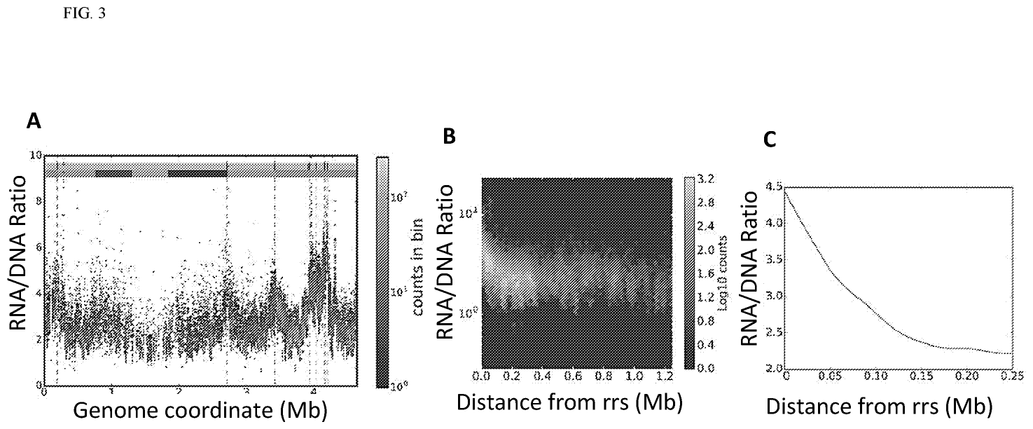

The seven ribosomal RNA operons in the E. coli genome are located within each of the major peaks of transcriptional propensity (FIG. 3A). By subtracting the LOWESS local regression smoothing on transcriptional propensity with distance from a ribosomal RNA operon (rrn), from the overall transcriptional propensity signal, the major waveform pattern is mostly eliminated, while local peaks & troughs are still apparent.

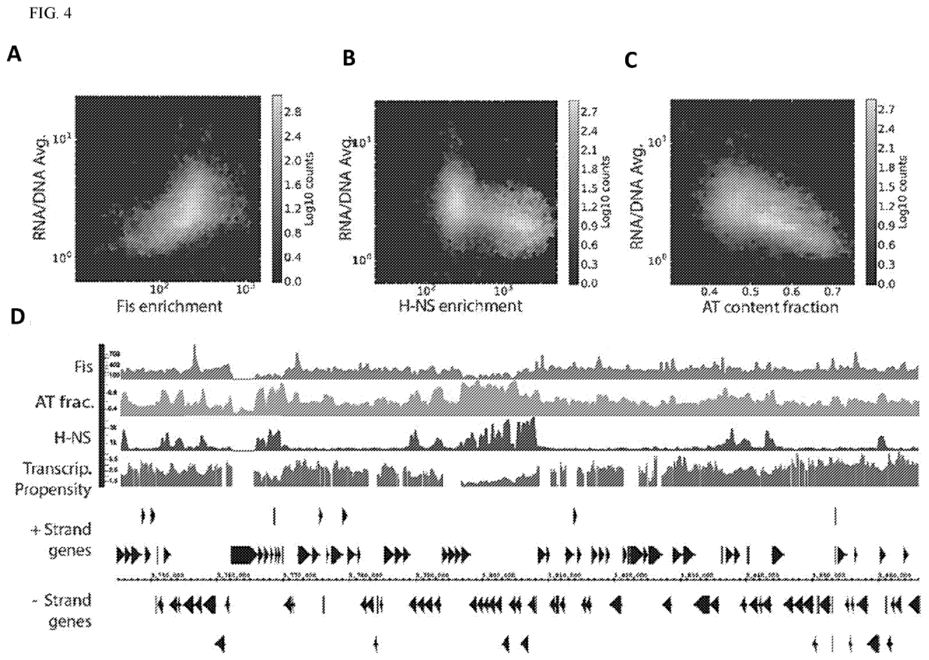

The correlation of transcriptional propensity with several characterized genomic features was examined using rolling-window medians over 500 bp for each data set. Despite the fact that the abundant NAP Fis is not expected to bind the reporter construct, transcriptional propensity is highly positively correlated with Fis binding level at genomic integration sites (Spearman ρ=0.5, FIG. 4A). Conversely, transcriptional propensity is strongly negatively correlated with H-NS binding (Spearman ρ=0.58, FIG. 4B). (Kahramanoglou et al. 2011). These findings are consistent with a heterochromatin-like gene silencing role for H-NS. Transcriptional propensity is also negatively correlated with protein occupancy, as it is significantly lower in tsEPODS than in other sites, strongly supporting reporter silencing observed by Bryant et al. when integrated within tsEPODs (Bryant et al. 2014a; Vora, Hottes, and Tavazoie 2009a).

RNA abundance from native genes displays only a weak positive correlation with transcriptional propensity (Spearman ρ=0.23). However, when larger rolling median windows are used for RNA abundance from native genes, correlation with transcriptional propensity is much higher (Spearman ρ=0.51). These results show that while highly expressed genes are more frequently located in high transcriptional propensity regions, the regulatory logic governing expression of individual genes is dominant over the underlying transcriptional propensity of a given region.

The correlation of neighboring RNA abundance in all orientations relative to the reporter on transcriptional propensity was examined. Native RNA abundances resulting from tandem orientation (or co-directional) transcription with reporters have weak correlation with transcriptional propensity. These data indicate that even very high neighboring transcription may have only a mild impact on transcriptional propensity. Since the correlations of native RNA abundances with transcriptional propensity in the tandem orientation with respect to the reporter are very similar regardless of which is upstream, insulation by the strong upstream transcriptional terminator of the reporter is also validated. Correlation of transcriptional propensity with RNA abundance from the divergent or convergent orientation is lower compared to tandem orientations. Together, these results indicate that neighboring transcription mildly effects transcriptional propensity. These results are largely consistent with the careful mechanistic studies of orientation-dependent expression interference between genes on plasmids (Yeung et al. 2017b). Transcriptional propensity from reporters on each strand display the same overall waveform pattern and are highly correlated. No evidence for a strand bias depending on the reporter direction with respect to replication direction was observed.

Binding of other NAPs (HU, LRP and SeqA) was not well correlated with transcriptional propensity nor was RNAP binding. No correlation of transcriptional propensity with a measure of DNA supercoiling density or with reporter location with respect to genes encodeding proteins recognized by the signal recognition particle was observed. Mean adenine and thiamine (AT) content in a 500 bp window around insertion locations was strongly negatively correlated with transcriptional propensity. AT content is also highly correlated with H-NS and protein occupancy binding. For this reason, conditional mutual information analysis was used to rank features for predicting transcriptional propensity. Together, these features describe 69.9% of the transcriptional propensity variation.

iPAGE analysis was used to identify pathways (Gene Ontology annotations) that are significantly informative about the transcriptional propensity at each gene location (Goodarzi, Elemento, and Tavazoie 2009). Large ribosomal subunit genes are significantly informative of high transcriptional propensity (GO:0022625). Genes in pathways for enterobacterial common antigen biosynthesis or organic phosphonate catabolism are clustered together in high transcriptional propensity peaks. Cellular amino acid biosynthetic process (GO:0008652), which has 105 genes in E. coli, is also significantly predictive of high transcriptional propensity. Intracellular protein transmembrane transport (GO:0065002), which is composed of twin-arginine transport genes for the export of folded proteins and also genes for SRP-dependent cotranslational protein targeting machinery, which are not clustered together were also identified. However, there is no difference in local transcriptional propensity between genes that encode products that are recognized by SRP and all other genes.

The correlation between integration density and multiple known genomic features was also tested. A b-spline was used to transform integration density over 500 bp rolling window signal to correct for expected differences in integrations densities that result from high gene dosage near the origin during transformation and Tn5 integration. H-NS binding had a very strong positive correlation with integration density. In contrast, low gene expression from eukaryotic genomes is well-correlated with heterochromatin that is inaccessible to transposon insertion. The correlation with H-NS was higher than the positive correlation of integration density with AT-content.

Table 1 shows exemplary regions that led to high levels of expression of inserted genes. The peaks ranges were identified by the shortest possible length that encompassed a region of high transcriptional propensity above the regional median propensity. These ranges comprise extended regions (>5 kb) which together make up 4.4% of the genome with the highest transcriptional propensity.

TABLE 1

| | | | | | | | intergenic | max |
|---|---|---|---|---|---|---|---|---|
| Average | start | end | length | center | maximum | minimum | center | coord |
| 29.4 | 281404 | 288692 | 7288 | 285048 | 52.9 | 20.9 | 285168 | 285070 |
| 18 | 4189573 | 4204803 | 15230 | 4197188 | 24.6 | 13.9 | 4199462 | 4200392 |
| 15.2 | 4128474 | 4165334 | 36860 | 4146904 | 22.1 | 10.14 | 4158225 | 4144823 |
| 13.9 | 4003592 | 4060559 | 56967 | 4032075.5 | 24.5 | 6.2 | 4042031 | 4041973 |
| 14.6 | 3949128 | 3980248 | 31120 | 3964688 | 24 | 10.2 | 3965702 | 3965302 |
| 15.3 | 4313204 | 4318232 | 5028 | 4315718 | 23.8 | 9.55 | 4314301 | 4315234 |
| 10.4 | 3387507 | 3439571 | 52064 | 3413539 | 18.1 | 5.5 | 3390503 | 3408955 |

### Example 2

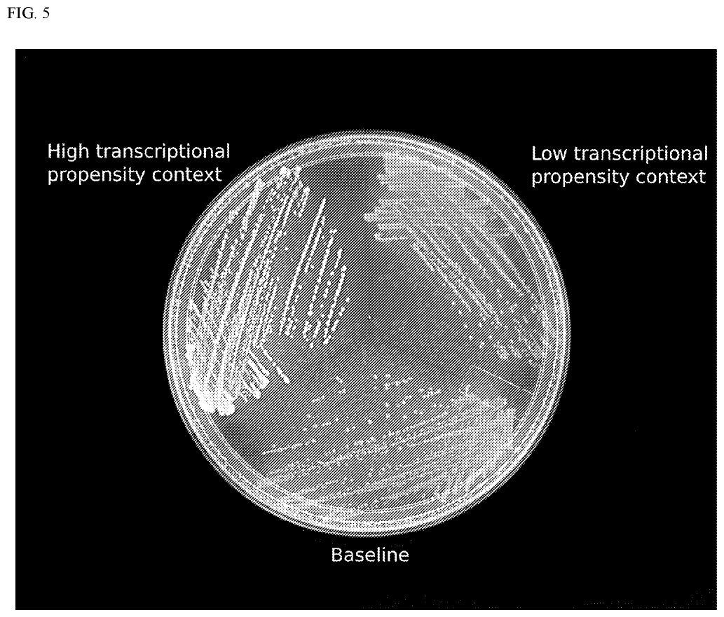

Dozens of existing genomic features from the genomic regions described in Example 1, such as protein binding and genome conformation contact maps, were compared to determine features responsible for the substantial expression variation. Of all genomic features, the proportion of guanine (G) and cytosine (C) in the overall nucleobase composition was the most strongly correlated with transcriptional propensity. Additionally, it was observed that long stretches of extreme GC content have particularly extreme transcription differences. Based on these observations, a synthetic DNA construct was constructed with the exact same reporter construct flanked by 3.5 kilobases of DNA with extremely low (35%) or high (65%) GC content, obtained from the genomes of different organisms (S. cerevisiae and H sapiens, respectively). Two plasmids that were identical with the exception of the GC content flanking the reporter construct were transformed into the E. coli strain Top10. The fluorescence produced by the 65% GC content plasmid was very strong compared to the 35% plasmid, which does not appear to have a fluorescent signal compared to a strain that expresses a repressor for the reporter (FIG. 5). These results are consistent with the strong correlation of high GC content with transcriptional propensity.

### REFERENCES

Akhtar, Waseem, Johann de Jong, Alexey V. Pindyurin, Ludo Pagie, Wouter Meuleman, Jeroen de Ridder, Anton Berns, Lodewyk F. A. Wessels, Maarten van Lohuizen, and Bas van Steensel. 2013. “Chromatin Position Effects Assayed by Thousands of Reporters Integrated in Parallel.” Cell 154 (4): 914-27.
Baba, Tomoya, Takeshi Ara, Miki Hasegawa, Yuki Takai, Yoshiko Okumura, Miki Baba, Kirill A. Datsenko, Masaru Tomita, Barry L. Wanner, and Hirotada Mori. 2006. “Construction of Escherichia Coli K-12 in-Frame, Single-Gene Knockout Mutants: The Keio Collection.” Molecular Systems Biology 2 (February): 2006.0008.
Bakshi, Somenath, Heejun Choi, and James C. Weisshaar. 2015. “The Spatial Biology of Transcription and Translation in Rapidly Growing Escherichia Coli.” Frontiers in Microbiology 6 (July): 636.
Beckwith, J. R., E. R. Signer, and W. Epstein. 1966. “Transposition of the Lac Region of E. Coli.” Cold Spring Harbor Symposia on Quantitative Biology 31 (0): 393-401. Blattner, F. R. 1997. “The Complete Genome Sequence of Escherichia Coli K-12.” Science 277 (5331): 1453-62.
Block, Dena H. S., Razika Hussein, Lusha W. Liang, and Han N. Lim. 2012a. “Regulatory Consequences of Gene Translocation in Bacteria.” Nucleic Acids Research 40 (18): 8979-92.
2012b. “Regulatory Consequences of Gene Translocation in Bacteria.” Nucleic Acids Research 40 (18): 8979-92.
Bokal, A. J., 4th, W. Ross, and R. L. Gourse. 1995. “The Transcriptional Activator Protein FIS: DNA Interactions and Cooperative Interactions with RNA Polymerase at the Escherichia Coli rrnB P1 Promoter.” Journal of Molecular Biology 245 (3): 197-207.
Brambilla, Elisa, and Bianca Sclavi. 2015. “Gene Regulation by H-NS as a Function of Growth Conditions Depends on Chromosomal Position in Escherichia Coli.” G3 5 (4): 605-14.
Bryant, Jack A., Laura E. Sellars, Stephen J. W. Busby, and David J. Lee. 2014a. “Chromosome Position Effects on Gene Expression in Escherichia Coli K-12.” Nucleic Acids Research 42 (18): 11383-92.
2014b. “Chromosome Position Effects on Gene Expression in Escherichia Coli K-12.” Nucleic Acids Research 42 (18): 11383-92.
Cabrera, Julio E., and Ding J. Jin. 2006a. “Active Transcription of rRNA Operons Is a Driving Force for the Distribution of RNA Polymerase in Bacteria: Effect of Extrachromosomal Copies of rrnB on the in Vivo Localization of RNA Polymerase.” Journal of Bacteriology 188 (11): 4007-14.
2006b. “Active Transcription of rRNA Operons Is a Driving Force for the Distribution of RNA Polymerase in Bacteria: Effect of Extrachromosomal Copies of rrnB on the in Vivo Localization of RNA Polymerase.” Journal of Bacteriology 188 (11): 4007-14.
Chai, Qian, Bhupender Singh, Kristin Peisker, Nicole Metzendorf, Xueliang Ge, Santanu Dasgupta, and Suparna Sanyal. 2014. “Organization of Ribosomes and Nucleoids in Escherichia Coli Cells during Growth and in Quiescence.” The Journal of Biological Chemistry 289 (16): 11342-52.
Chen, Huiyi, Katsuyuki Shiroguchi, Hao Ge, and Xiaoliang Sunney Xie. 2015. “Genome-Wide Study of mRNA Degradation and Transcript Elongation in Escherichia Coli.” Molecular Systems Biology 11 (5): 808.
Chen, Ying-Ja, Peng Liu, Alec A. K. Nielsen, Jennifer A. N. Brophy, Kevin Clancy, Todd Peterson, and Christopher A. Voigt. 2013a. “Characterization of 582 Natural and Synthetic Terminators and Quantification of Their Design Constraints.” Nature Methods 10 (7): 659-64.
2013b. “Characterization of 582 Natural and Synthetic Terminators and Quantification of Their Design Constraints.” Nature Methods 10 (7): 659-64.
Clavel, Damien, Guillaume Gotthard, David von Steffen, Daniele De Sanctis, Helene Pasquier, Gerard G. Lambert, Nathan C. Shaner, and Antoine Royant. 2016. “Structural Analysis of the Bright Monomeric Yellow-Green Fluorescent Protein mNeonGreen Obtained by Directed Evolution.” Acta Crystallographica. Section D, Structural Biology 72 (Pt 12): 1298-1307.
Cooper, Stephen, and Charles E. Helmstetter. 1968. “Chromosome Replication and the Division Cycle of Escherichia Coli.” Journal of Molecular Biology 31 (3): 519-40. Dorman, Charles J. 2006. “DNA Supercoiling and Bacterial Gene Expression.” Science Progress 89 (3): 151-66.
Espah Borujeni, Amin, Anirudh S. Channarasappa, and Howard M. Salis. 2014. “Translation Rate Is Controlled by Coupled Trade-Offs between Site Accessibility, Selective RNA Unfolding and Sliding at Upstream Standby Sites.” Nucleic Acids Research 42 (4): 2646-59.
Fang, Ferric C., and Sylvie Rimsky. 2008. “New Insights into Transcriptional Regulation by H-NS.” Current Opinion in Microbiology 11 (2): 113-20.
French, S. L., and O. L. Miller Jr. 1989. “Transcription Mapping of the Escherichia Coli Chromosome by Electron Microscopy.” Journal of Bacteriology 171 (8): 4207-16. Gaal, Tamas, Benjamin P. Bratton, Patricia Sanchez-Vazquez, Alexander Sliwicki, Kristine Sliwicki, Andrew Vegel, Rachel Pannu, and Richard L. Gourse. 2016. “Colocalization of Distant Chromosomal Loci in Space in E. Coli: A Bacterial Nucleolus.” Genes & Development 30 (20): 2272-85.
Gao, Yunfeng, Yong Hwee Foo, Ricksen S. Winardhi, Qingnan Tang, Jie Yan, and Linda J. Kenney. 2017. “Charged Residues in the H-NS Linker Drive DNA Binding and Gene Silencing in Single Cells.” Proceedings of the National Academy of Sciences 114 (47): 12560-65.
Girgis, Hany S., Yirchung Liu, William S. Ryu, and Saeed Tavazoie. 2007a. “A Comprehensive Genetic Characterization of Bacterial Motility.” PLoS Genetics 3 (9): 1644-60.
2007b. “A Comprehensive Genetic Characterization of Bacterial Motility.” PLoS Genetics 3 (9): 1644-60.
Goodarzi, Hani, Olivier Elemento, and Saeed Tavazoie. 2009. “Revealing Global Regulatory Perturbations across Human Cancers.” Molecular Cell 36 (5): 900-911.
Hanahan, Douglas, Joel Jessee, and Fredric R. Bloom. 1991. “[4] Plasmid Transformation of Escherichia Coli and Other Bacteria.” In Methods in Enzymology, 63-113.
Higashi, Koichi, Toni Tobe, Akinori Kanai, Ebru Uyar, Shu Ishikawa, Yutaka Suzuki, Naotake Ogasawara, Ken Kurokawa, and Taku Oshima. 2016. “H-NS Facilitates Sequence Diversification of Horizontally Transferred DNAs during Their Integration in Host Chromosomes.” PLoS Genetics 12 (1): e1005796.
Hirvonen, C. A., W. Ross, C. E. Wozniak, E. Marasco, J. R. Anthony, S. E. Aiyar, V. H. Newburn, and R. L. Gourse. 2001. “Contributions of UP Elements and the Transcription Factor FIS to Expression from the Seven Rrn P1 Promoters in Escherichia Coli.” Journal of Bacteriology 183 (21): 6305-14.
Jeong, Da-Eun, Younju So, Soo-Young Park, Seung-Hwan Park, and Soo-Keun Choi. 2018. “Random Knock-in Expression System for High Yield Production of Heterologous Protein in Bacillus Subtilis.” Journal of Biotechnology 266 (January): 50-58.
Jin, Ding Jun, and Julio E. Cabrera. 2006. “Coupling the Distribution of RNA Polymerase to Global Gene Regulation and the Dynamic Structure of the Bacterial Nucleoid in Escherichia Coli.” Journal of Structural Biology 156 (2): 284-91.
Kahramanoglou, Christina, Aswin S. N. Seshasayee, Ana I. Prieto, David Ibberson, Sabine Schmidt, Jurgen Zimmermann, Vladimir Benes, Gillian M. Fraser, and Nicholas M. Luscombe. 2011. “Direct and Indirect Effects of H-NS and Fis on Global Gene Expression Control in Escherichia Coli.” Nucleic Acids Research 39 (6): 2073-91.
Klappenbach, J. A., J. M. Dunbar, and T. M. Schmidt. 2000. “rRNA Operon Copy Number Reflects Ecological Strategies of Bacteria.” Applied and Environmental Microbiology 66 (4): 1328-33.
Kosuri, Sriram, Daniel B. Goodman, Guillaume Cambray, Vivek K. Mutalik, Yuan Gao, Adam P. Arkin, Drew Endy, and George M. Church. 2013a. “Composability of Regulatory Sequences Controlling Transcription and Translation in Escherichia Coli.” Proceedings of the National Academy of Sciences of the United States of America 110 (34): 14024-29.
2013b. “Composability of Regulatory Sequences Controlling Transcription and Translation in Escherichia Coli.” Proceedings of the National Academy of Sciences of the United States of America 110 (34): 14024-29.
Lal, Avantika, Amlanjyoti Dhar, Andrei Trostel, Fedor Kouzine, Aswin S. N. Seshasayee, and Sankar Adhya. 2016. “Genome Scale Patterns of Supercoiling in a Bacterial Chromosome.” Nature Communications 7: 11055.
Le, Tung B. K., Maxim V. Imakaev, Leonid A. Mirny, and Michael T. Laub. 2013a. “High-Resolution Mapping of the Spatial Organization of a Bacterial Chromosome.” Science 342 (6159): 731-34.
2013b. “High-Resolution Mapping of the Spatial Organization of a Bacterial Chromosome.” Science 342 (6159): 731-34.
Lioy, Virginia S., Axel Cournac, Martial Marbouty, Stéphane Duigou, Julien Mozziconacci, Olivier Espéli, Frederic Boccard, and Romain Koszul. 2018a. “Multiscale Structuring of the E. Coli Chromosome by Nucleoid-Associated and Condensin Proteins.” Cell 172 (4): 771-83.e18.
2018b. “Multiscale Structuring of the E. Coli Chromosome by Nucleoid-Associated and Condensin Proteins.” Cell 172 (4): 771-83.e18.
Marbouty, Martial, Antoine Le Gall, Diego I. Cattoni, Axel Cournac, Alan Koh, Jean-Bernard Fiche, Julien Mozziconacci, Heath Murray, Romain Koszul, and Marcelo Nollmann. 2015. “Condensin- and Replication-Mediated Bacterial Chromosome Folding and Origin Condensation Revealed by Hi-C and Super-Resolution Imaging.” Molecular Cell 59 (4): 588-602.
Martinez-Antonio, Agustino, Alejandra Medina-Rivera, and Julio Collado-Vides. 2009. “Structural and Functional Map of a Bacterial Nucleoid.” Genome Biology 10 (12): 247. Paul, Brian J., Wilma Ross, Tamas Gaal, and Richard L. Gourse. 2004. “rRNA Transcription in Escherichia Coli.” Annual Review of Genetics 38: 749-70.
Rainey, F. A., N. L. Ward-Rainey, P. H. Janssen, H. Hippe, and E. Stackebrandt. 1996. “Clostridium Paradoxum DSM 7308T Contains Multiple 16S rRNA Genes with Heterogeneous Intervening Sequences.” Microbiology 142 (Pt 8) (August): 2087-95.
Ross, W., J. F. Thompson, J. T. Newlands, and R. L. Gourse. 1990. “E. coli Fis Protein Activates Ribosomal RNA Transcription in Vitro and in Vivo.” The EMBO Journal 9 (11): 3733-42.
Sousa, C., V. de Lorenzo, and A. Cebolla. 1997. “Modulation of Gene Expression through Chromosomal Positioning in Escherichia Coli.” Microbiology 143 (Pt 6) (June): 2071-78. Vora, Tiffany, Alison K. Hones, and Saeed Tavazoie. 2009a. “Protein Occupancy Landscape of a Bacterial Genome.” Molecular Cell 35 (2): 247-53.
2009b. “Protein Occupancy Landscape of a Bacterial Genome.” Molecular Cell 35 (2): 247-53.
Wang, Xindan, Tung B. K. Le, Bryan R. Lajoie, Job Dekker, Michael T. Laub, and David Z. Rudner. 2015. “Condensin Promotes the Juxtaposition of DNA Flanking Its Loading Site in Bacillus Subtilis.” Genes & Development 29 (15): 1661-75.
Yeung, Enoch, Aaron J. Dy, Kyle B. Martin, Andrew H. Ng, Domitilla Del Vecchio, James L. Beck, James J. Collins, and Richard M. Murray. 2017a. “Biophysical Constraints Arising from Compositional Context in Synthetic Gene Networks.” Cell Systems 5 (1): 11-24.e12.
2017b. “Biophysical Constraints Arising from Compositional Context in Synthetic Gene Networks.” Cell Systems 5 (1): 11-24.e12.

All publications, patents, patent applications and accession numbers mentioned in the above specification are herein incorporated by reference in their entirety. Although the invention has been described in connection with specific embodiments, it should be understood that the invention as claimed should not be unduly limited to such specific embodiments. Indeed, various modifications and variations of the described compositions and methods of the invention will be apparent to those of ordinary skill in the art and are intended to be within the scope of the following claims.

1. An E. coli bacterium comprising a recombination target site and/or heterologous gene at one or more genomic positions comprising at least one expression enhancement sequence.

2. The bacterium of claim 1, wherein said expression enhancement sequence is at least 50% GC nucleotides.

3. The bacterium of claim 2, wherein said expression enhancement sequence is at least 65% GC nucleotides.

4. The bacterium of claim 1, wherein said recombination target site and/or heterologous genes is flanked on at least one side by said expression enhancement sequence.

5. The bacterium of claim 1, wherein said recombination target site and/or heterologous genes is flanked on both sides by said expression enhancement sequence.

6. The bacterium of claim 1, wherein said expression enhancement sequence is at least 500 bp per side.

7. The bacterium of claim 1, wherein said expression enhancement sequence is at least 1000 bp per side.

8. The bacterium of claim 1, wherein said expression enhancement sequence is one or more genomic positions selected from the group consisting of nucleotides 281404 to 288692, 4189573 to U.S. Pat. Nos. 4,204,803, 4,128,474 to U.S. Pat. Nos. 4,165,334, 4,003,592 to U.S. Pat. Nos. 4,060,559, 3,949,128 to 3980248, 4313204 to U.S. Pat. Nos. 4,318,232, and 3,387,507 to 3439571 and sequences at least 90% homologous to said genomic positions.

9. The bacterium of claim 1, wherein said expression enhancement sequence is one or more genomic positions selected from the group consisting of nucleotides 281404 to 288692, 4189573 to U.S. Pat. Nos. 4,204,803, 4,128,474 to U.S. Pat. Nos. 4,165,334, 4,003,592 to U.S. Pat. Nos. 4,060,559, 3,949,128 to U.S. Pat. Nos. 3,980,248, 4,313,204 to U.S. Pat. Nos. 4,318,232, and 3,387,507 to 3439571.

10-11. (canceled)

12. The bacterium of claim 1, wherein said recombination target site is a site-specific recombination target site.

13. The bacterium of claim 12, wherein said site-specific recombination target site is selected from the group consisting of FLP recombination target (FRT), LOX, attP/B recognition sites, gamma-delta resolvase site, Tn3 resolvase site, lambda-red recombination, Clustered regularly interspaced short palindromic repeats (CRISPR), and φC31 integrase target site.

14. The bacterium of claim 1, wherein said recombination target site further comprises one or more elements selected from the group consisting of a promoter, a repressor, a reporter construct, and a purification tag gene.

15. The bacterium of claim 14, wherein said promoter is an inducible promoter.

16. The bacterium of claim 14, wherein said promoter is selected from the group consisting of a lac promoter, a tac promoter, and a T7 promoter.

17. The bacterium of claim 1, wherein said bacterium expresses a gene of interest inserted into said chromosome at said genomic position at a higher level relative to the level of expression of said gene interested into a different location on the said chromosome.

18. The bacterium of claim 17, wherein said gene of interest is expressed at a level of at least 2 fold relative to the level of expression of said gene interested into a different location on said chromosome.

19. The bacterium of claim 18, wherein said gene of interest is expressed at a level of at least 10 fold relative to the level of expression of said gene interested into a different location on said chromosome.

20. A kit or system, comprising:
a) the bacterium of claim 1; and
b) a recombination enzyme specific for said recombination target or a nucleic acid encoding said recombination enzyme.

21. (canceled)

22. The kit or system of claim 20, wherein said nucleic acid encoding said recombination enzyme is on a plasmid or incorporated into the chromosome of said bacterium.

23. A method of expressing a gene of interest, comprising:
a) contacting a nucleic acid encoding said gene of interest with the bacterium of claim 1 under conditions such that said nucleic acid integrates into the chromosome of said bacterium at said genomic positions; and
b) expressing said gene of interest.

24-28. (canceled)


SEQUENCE LISTING (3 sequences)

SEQ ID NO: 1 - DNA, 34 bp, Artificial sequence
  Synthetic
gaagttcctattctctagaaagtataggaacttc

SEQ ID NO: 2 - DNA, 13 bp, Artificial sequence
  Synthetic
gaagttcctattc

SEQ ID NO: 3 - DNA, 34 bp, Artificial sequence
  Synthetic
  misc_feature (14)..(16): n is a, c, g, or t
  misc_feature (19)..(21): n is a, c, g, or t
ataacttcgtatannntannntatacgaagttat